(19) United States
(12) Patent Application Publication
(10) Pub. No.: US 2023/0268566 A1
(43) Pub. Date: Aug. 24, 2023

(54) POSITIVE ELECTRODE ACTIVE MATERIAL, POSITIVE ELECTRODE INCLUDING THE SAME, SECONDARY BATTERY INCLUDING THE SAME, AND GAS ANALYZING APPARATUS

(71) Applicant: SK On Co., Ltd., Seoul (KR)

(72) Inventors: Kyung Bin YOO, Daejeon (KR); Duck Chul HWANG, Daejeon (KR)

(21) Appl. No.: 18/093,813

(22) Filed: Jan. 6, 2023

(30) Foreign Application Priority Data
Jan. 14, 2022 (KR) 10-2022-0006085

(51) Int. Cl. H01M 10/42; G01N 33/00; H01M 4/525; H01M 10/052

(52) U.S. Cl. CPC H01M 10/4285; G01N 33/0027; H01M 4/525; H01M 10/052; H01M 2004/028

(57) ABSTRACT

A positive electrode active material has a pressure of gas produced by a reaction with an electrolyte solution of 0.4 to 0.6 atm/mAh. The positive electrode active material according to the present disclosure allows prediction of an amount of gas produced and gas components in a secondary battery cell without actually manufacturing a secondary battery cell. In addition, a process from sample preparation to measurement completion, which is required for measuring an amount of gas produced, may be performed within a short time.

### CROSS-REFERENCE TO RELATED APPLICATION(S)

This application claims benefit of priority to Korean Patent Application No. 10-2022-0006085 filed on Jan. 14, 2022 in the Korean Intellectual Property Office, the disclosure of which is incorporated herein by reference in its entirety.

### BACKGROUND

### 1. Field

The present disclosure relates to a positive electrode active material, a positive electrode including the same, a secondary battery including the same, and a gas analyzing apparatus capable of using pressure and characteristics of gas produced by the positive electrode active material for analysis.

### 2. Description of Related Art

A secondary battery is a battery which may be used repeatedly in a charging process and in a reverse direction, with discharge, converting chemical energy into electrical energy, and a nickel-cadmium (Ni—Cd) battery, a nickel-hydrogen (Ni-MH) battery, a lithium-metal battery, a lithium-ion (Ni-ion) battery, a lithium-ion polymer battery, and the like belong to the secondary battery. Among the secondary batteries, a lithium secondary battery having high energy density and voltage, a long cycle life, and a low self-discharge rate has been commercialized and is widely used.

A lithium secondary battery has a risk of ignition and explosion when exposed to a high temperature. In addition, when a large current flows within a short time due to overcharge, external short circuit, nail penetration, local crush, and the like, the battery has a risk of ignition and explosion as it is heated by heat. As an example, as a result of a reaction between an electrolyte solution and an electrode, gas is produced to increase battery internal pressure, and thus, a lithium secondary battery may explode at a specific pressure or more.

Depending on the reaction in the lithium secondary battery, various kinds of gases such as carbon dioxide, carbon monoxide, and hydrogen may be produced. Internally produced gas such as carbon dioxide may be in a reversible state in which it may revert to an original material while being charged depending on conditions, but largely remains in a gaseous state in a battery to increase internal pressure and cause a swelling phenomenon to inflate the battery. The swollen battery has an increased thickness, so that it may not be mounted well in electronic electrical equipment which is designed to be equipped with a battery, or may be determined to be defective due to a bulging appearance thereof to lose value as a commodity.

Meanwhile, according to a recent trend of growing interest of electric automobiles, gas production during the use of a lithium secondary battery is emerging as an important issue related to the safety of an electric automobile as well as battery life. While an attempt to increase the capacity of a lithium secondary battery continues for increasing the mileage of an electric automobile, a gas production issue increases also together.

Accordingly, various attempts to measure and quantify the gas production of a lithium secondary battery are being made. In general, a method of measuring gas from a lithium secondary battery is performed by manufacturing a secondary battery cell, evaluating life, high-temperature storage characteristics, and the like of the cell, perforate the cell, collecting gas, and investigating the amount and the components of the collected gas. As another approach, after a secondary battery cell is manufactured, the life, the high-temperature storage characteristics, and the like of the cell are evaluated, the secondary battery cell is immersed in a liquid, and a volume change of the secondary battery cell is measured to indirectly quantify produced gas.

However, such conventional gas analysis methods may perform analysis only in a state of gas produced after a secondary battery cell is actually manufactured and evaluation is performed, and thus, there is a difficulty in terms of time and costs. In addition, since the secondary battery cell includes various materials such as a positive electrode, a negative electrode, an electrolyte solution, and a separator, it is not easy to analyze the case of gas production and find an improvement plan therefor.

### SUMMARY

An aspect of the present disclosure may provide a positive electrode active material which may guarantee a small amount of gas produced without actually manufacturing a full-cell, and a positive electrode and a secondary battery including the same.

Another aspect of the present disclosure may provide a gas analyzing apparatus which allows measurement of an amount of gas produced and gas components without actually manufacturing a secondary battery full-cell, and may perform a process from sample preparation to measurement completion within a short time.

According to an aspect of the present disclosure, a positive electrode active material having a pressure of gas produced by a reaction with an electrolyte solution of 0.4 to 0.6 atm/mAh is provided.

The reaction may be performed at a temperature of 70 to 75° C.

The positive electrode active material may be collected from a half-cell charged with predetermined SOC.

The positive electrode active material may include 80 mol% or more of nickel.

A weight ratio between the positive electrode active material and the electrolyte solution may be 1:1 to 3:1.

According to another aspect of the present disclosure, a positive electrode includes the positive electrode active material.

According to another aspect of the present disclosure, a secondary battery includes: the positive electrode; a negative electrode; and a separator interposed between the positive electrode and the negative electrode.

According to another aspect of the present disclosure, a gas analyzing apparatus includes: a lower plate including a retention portion in which an electrolyte solution and a positive electrode active material are retained; an upper plate including a first flow path in which gas produced by a reaction of the electrolyte solution and the positive electrode active material moves and a pressure measuring sensor; an internal pressure control port controlling opening and closing of a second flow path communicating with the first flow path of the upper plate and controls pressure inside the apparatus; and a sealing member sealing a space between the upper plate and the lower plate.

The retention portion may have a volume of 70 to 80 mm³.

An oven in which the gas analyzing apparatus is loaded may be further included.

A data collection port which collects data measured from the gas analyzing apparatus may be further included.

A fastening member which fixes the upper plate and the lower plate may be further included.

A gas chromatography analysis unit which is connected to the internal pressure control port may be further included.

A temperature sensor which measures a reaction temperature of the electrolyte solution and the positive electrode active material may be further included.

The temperature sensor may be formed on a lower surface of the upper plate adjacent to the retention portion.

The lower surface of the upper plate adjacent to the retention portion may be formed in a dome structure.

The positive electrode active material may be collected from a half-cell charged with predetermined SOC.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 schematically illustrates a gas analyzing apparatus according to an exemplary embodiment in the present disclosure;

FIG. 2 is a top view of a lower plate of the gas analyzing apparatus according to an exemplary embodiment in the present disclosure;

FIG. 3 schematically illustrates a gas analyzing apparatus according to another exemplary embodiment in the present disclosure;

FIG. 4 schematically illustrates a gas analyzing apparatus according to another exemplary embodiment in the present disclosure;

FIG. 5 is a schematic diagram schematically illustrating a process in which an electrolyte solution reacts with a charged positive electrode active material on a surface of the positive electrode active material to produce gas;

FIG. 6 schematically illustrates a method of analyzing gas using the gas analyzing apparatus according to an exemplary embodiment in the present disclosure; and

FIG. 7 is a graph illustrating results of measuring gas pressure according to Examples 1 to 3 of the present disclosure.

### DETAILED DESCRIPTION

Hereinafter, exemplary embodiments in the present disclosure will now be described in detail with reference to various examples. However, the exemplary embodiments of the present disclosure may be modified in many different forms and the scope of the disclosure should not be limited to the embodiments set forth herein.

According to an aspect of the present disclosure, a positive electrode active material which has a pressure of gas produced by a reaction with an electrolyte solution is 0.4 to 0.6 atm/mAh is provided. Though there is an increasing trend in the use of a positive electrode active material having a high content of nickel (Ni) in order to provide a high-capacity lithium secondary battery, as a nickel content is higher, the positive electrode active material donates more electrons during charging, and thus, oxidizing power is further increased and the amount of gas produced is also increased. In addition, in order to measure the amount of gas, a secondary full-cell should be necessarily manufactured, and thus, costs and time are consumed. The inventors of the present disclosure found that when a positive electrode active material having a pressure of gas produced by a reaction with an electrolyte solution in a range of 0.4 to 0.6 atm/mAh is used, as the pressure value is lower in the range, the amount of gas produced in a real full-cell is small, thereby completing the present disclosure.

Due to the high oxidizing power of the surface of a charged positive electrode active material, an electrolyte solution is oxidized to produce gas, and thus, the positive electrode active material may be collected from a charged half-cell charged with predetermined state of charge (SOC). For example, the positive electrode active material may be collected from a cell charged with SOC of 10% or more, 20% or more, 30% or more, 40% or more, or 50% or more and 100% or less, 90% or less, 80% or less, 70% or less, 60% or less, or 50% or less.

Without being particularly limited, the collection of the positive electrode active material may include: manufacturing a half-cell including the collected positive electrode active material; charging the half-cell to predetermined SOC to be desired; and drying an electrode obtained by disassembling the charged half-cell and then obtaining the positive electrode active material from the dried electrode.

It is preferred that the positive electrode active material includes 80 mol% or more of nickel. Since a positive electrode active material including less than 80 mol% of nickel has a very small amount of gas produced, measurement reliability may be lowered.

The electrolyte solution is not particularly limited as long as it may be applied to a secondary battery. For example, as the electrolyte solution, a solution of 1 M LiPF6 dissolved in a mixed solvent of EC/EMC/DEC (25/45/30; volume ratio) may be used, and, if necessary, an additive may be added thereto. In a range of a pressure of gas produced by the reaction of the electrolyte solution and the positive electrode active material of 0.4 to 0.6 atm/mAh, as a pressure value is low, the amount of gas produced in a real full-cell may be expected to be small.

It is preferred that the reaction of the positive electrode active material and the electrolyte solution is performed at a temperature of 70 to 75° C. Evaluation of full-cell storage at a high temperature which is generally performed is performed at about 60° C., and as a concept of acceleration evaluation, the gas pressure in the present disclosure may be performed at 70 to 75° C.

A weight ratio between the positive electrode active material and the electrolyte solution is preferably 1:1 to 3:1, and more preferably 1.5:1 to 2.5:1, in order to maximize a reaction area of the positive electrode active material and the electrolyte solution.

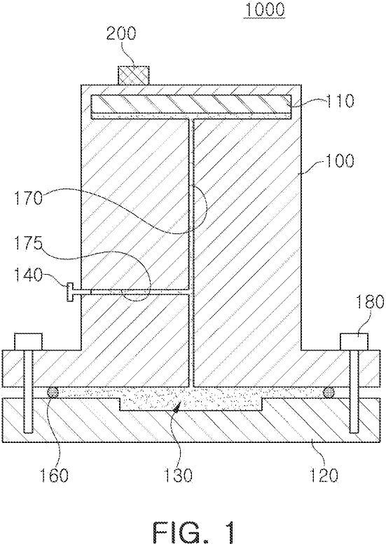

According to another aspect of the present disclosure, a gas analyzing apparatus which may analyze the pressure of gas produced from the reaction of a positive electrode active material and an electrolyte solution described above and other characteristics of gas is provided. When the apparatus of the present disclosure is used, an amount of gas produced and gas components in a secondary battery cell may be predicted without actually manufacturing a secondary battery cell full-cell, and a process from sample preparation to measurement completion required for measurement may be performed within a short time. FIG. 1 schematically illustrates a gas analyzing apparatus according to an exemplary embodiment in the present disclosure, and referring to FIG. 1, the present disclosure will be described in detail.

According to an aspect of the present disclosure, a gas analyzing apparatus 1000 including: a lower plate 120 including a retention portion 130 in which an electrolyte solution and a positive electrode active material are retained; an upper plate 100 including a first flow path 170 in which gas produced by a reaction of the electrolyte solution and the positive electrode active material moves and a pressure measuring sensor 110; an internal pressure control port 140 which controls opening and closing of a second flow path 175 communicating with the first flow path 170 of the upper plate 100 and controls pressure inside the apparatus; and a sealing member 160 sealing a space between the upper plate 100 and the lower plate 120 is provided.

The lower plate 120 may include the retention portion 130 in which a positive electrode active material to be analyzed and an electrolyte solution are retained. The shape of the retention portion 130 is not particularly limited, and for example, the lower plate 120 may include a groove formed on a part of the lower plate 120 and the groove may be the retention portion 130.

Without being particularly limited, the retention portion may have a volume of 70 to 80 mm³, and when out of the range, measurement reliability may be decreased or economic feasibility may be deteriorated.

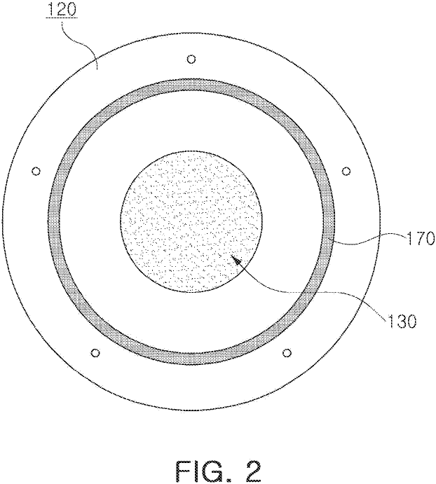

Meanwhile, FIG. 2 is a top view of the lower plate 120 of the gas analyzing apparatus 1000 according to an exemplary embodiment in the present disclosure. In the lower plate 120, a sealing groove in which the sealing member 160 for sealing a space between the upper plate 100 and the lower plate 120 to close the space so that gas produced from the positive electrode active material and the electrolyte solution may not be released to the outside other than a pressure measuring sensor 110 disposed on the upper plate 100 is interposed may be formed. The sealing member 160 may be a sealing member 160 made of O-ring or rubber which may maintain air tightness in a contact surface between the upper plate 100 and the lower plate 120.

Meanwhile, the shape of the lower plate 120 is not particularly limited. As shown in FIG. 2, it may have a circular section, but may be implemented in various shapes such as a quadrangle and a triangle, and is not limited to a particular shape.

The material of the lower plate 120 is not particularly limited, and for example, may be formed by including a metal such as aluminum and stainless steel.

The gas analyzing apparatus 1000 of the present disclosure may include the upper plate 100 including the first flow path 170 in which gas produced from the positive electrode active material and the electrolyte solution moves and the pressure measuring sensor 110. The first flow path 170 serves as a passage in which gas produced from the positive electrode active material and the electrolyte solution moves, and more specifically, gas produced from the positive electrode active material and the electrolyte solution of the retention portion 130 moves through the first flow path 170 formed inside the upper plate 100, and the pressure of the gas may be measured in the pressure measuring sensor 110 disposed on the upper plate 100. Though the first flow path 170 is not particularly limited, a “T” shape may be formed inside the upper plate 100, as shown in FIG. 1.

Inside the upper plate 100, the position where the pressure measuring sensor 110 is formed is not limited. However, the pressure measuring sensor 110 may be disposed to be in contact with the upper portion of the upper plate 100, as shown in FIG. 1, so that it is adjacent to a data collection port 200 described later considering the characteristics of gas of rising from the ground, but is not limited thereto.

The material of the upper plate 100 is not particularly limited, and for example, may be formed by including a metal such as aluminum and stainless steel.

The second flow path 175 communicating with the first flow path 170 may be included inside the upper plate 100. In addition, an internal pressure control port which controls opening and closure of the second flow path 175 may be formed on one surface of the upper plate. The internal pressure control port may serve to control pressure inside the apparatus, such as fastening the upper plate 100 and the lower plate 120 to integrate them and then removing pressure applied to the inside of the apparatus and matching the internal pressure to external pressure by opening and closing the second flow path 175, or removing pressure applied to the inside of the apparatus after measurement completion. For example, when gas analysis is performed several times using various positive electrode active materials and electrolyte solutions, initial pressure may become different, and it is necessary to reset initial pressure in the apparatus to normal pressure for accurate measurement even with small change in pressure. Therefore, after the upper plate 100 and the lower plate 120 is combined, the internal pressure control port may be opened to reset the internal pressure to normal pressure.

In addition, the second flow path 175 may be used as an inlet of a washing liquid for washing the gas analyzing apparatus 1000. For example, a solvent in the electrolyte solution is evaporated at a high temperature and adsorbed into the apparatus, unless the flow path, the pressure measuring sensor 110, and the like in the apparatus are not washed, the flow path may be blocked or sensor movement is impeded, so that measurement may not be performed well. When only the first flow path 170 is formed, a passage in which the washing liquid flows and a passage from which the washing liquid is released are the same, so that it may not be easy to wash inside, but when a separate second flow path 175 is formed, the washing liquid may flow in and be released more smoothly.

Meanwhile, according to another exemplary embodiment in the present disclosure, a gas chromatography analysis unit (not shown) connected to the internal pressure control port 140 may be further included. Accordingly, gas released through the second flow path 175 may be collected and the kind and the components of gas may be analyzed.

The combination method of the upper plate 100 and the lower plate 120 is not particularly limited. For example, the upper plate 100 and the lower plate 120 may be combined and fixed using a fastening member 180 such as screws, bolts, and nuts. The upper plate 100 and the lower plate 120 may be fastened and combined, using bolts and nuts disposed in holes formed outside the sealing member 160 of the lower plate 120 and the position of the upper plate 100 formed to correspond to the position of the sealing member 160, as shown in FIG. 1.

The gas analyzing apparatus 1000 of the present disclosure may include a data collection port 200 which collects data measured from the gas analyzing apparatus 1000. The data collection port 200 may receive pressure information measured from the pressure measuring sensor 110 in real time and record the information.

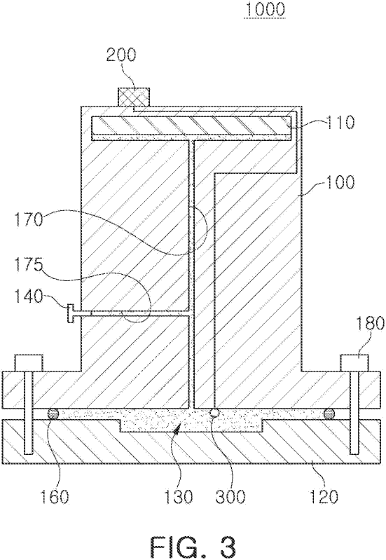

FIG. 3 schematically illustrates the gas analyzing apparatus 1000 according to another exemplary embodiment in the present disclosure, and according to another exemplary embodiment, the gas analyzing apparatus 1000 may further include a temperature sensor 300 which measure a reaction temperature of the positive electrode active material and the electrolyte solution. When the apparatus of the present disclosure is used, the measurement is performed in a state in which the entire apparatus is loaded in a heating means such as an oven 1500, but a temperature difference may occur depending on the position of the heating means, and in particular, since a temperature at which the positive electrode active material is actually reacted with the electrolyte solution to produce gas is important, it is preferred to include a temperature sensor 300 for measuring a reaction temperature and control it, for example, a thermal couple or the like. The temperature sensor is connected to the data collection port 200 which collects data measured from the gas analyzing apparatus 1000 and send the measured temperature information to the data collection port.

Meanwhile, it is more preferred that the temperature sensor is formed on the lower surface of the upper plate 100 adjacent to the retention portion 130, for more accurately measuring the reaction temperature of the positive electrode active material and the electrolyte solution.

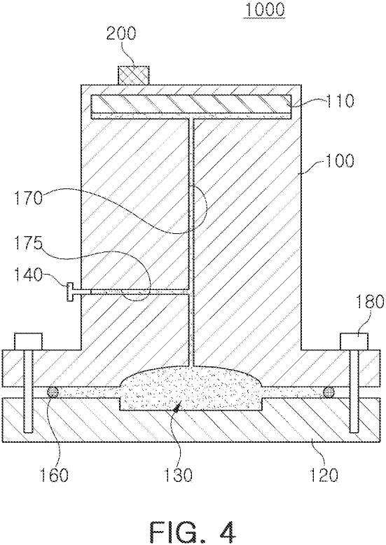

FIG. 4 schematically illustrates a gas analyzing apparatus 1000 according to another exemplary embodiment in the present disclosure, and according to another exemplary embodiment in the present disclosure, the lower surface of the upper plate 100 adjacent to the retention portion 130 may be formed in a dome structure.

Specifically, when the positive electrode active material and the electrolyte solution is retained in the retention portion 130 and the upper plate 100 and the lower plate 120 is combined, the diameter of a flow path to the pressure measuring sensor 100 is somewhat narrow, and due to the volume of the positive electrode active material and the electrolyte solution, the opening of the first flow path 170 toward the pressure measuring sensor 110 may be blocked by the positive electrode active material and the electrolyte solution. However, as shown in FIG. 4, when the lower surface of the upper plate 100 adjacent to the retention portion 130 is cut have a round part, that is, to have a dome shape, an increase in volume of internal space is increased and a contact between the positive electrode active material and the electrolyte solution is minimized, thereby preventing blockage of the inlet of the first flow path 170. Meanwhile, the groove of the lower plate 120 in which the retention portion 130 is formed may be formed to be deeper. However, in this case, an error in pressure measurement becomes large so that it is difficult to confirm a deviation, as compared with the case of forming the lower surface of the upper plate 100 adjacent to the retention portion 130 in a dome structure.

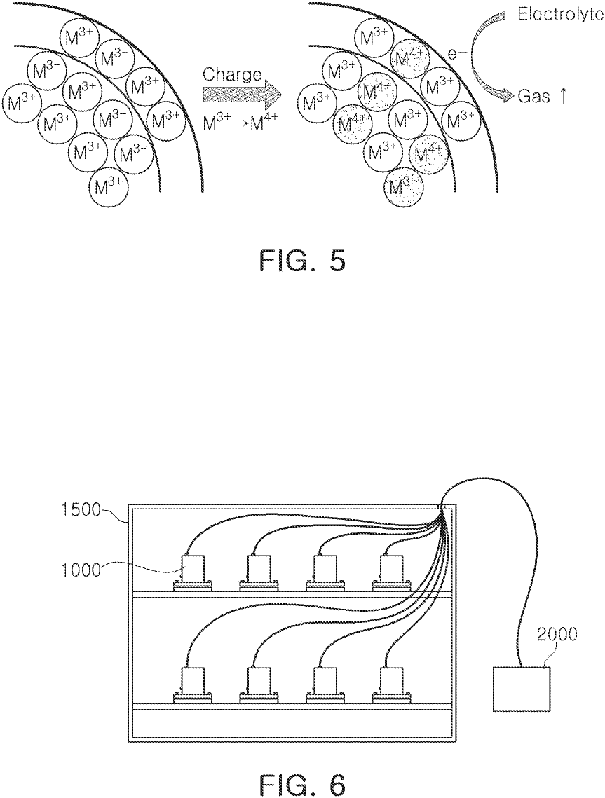

Meanwhile, as described above, a main cause of gas production in a lithium secondary battery is gas production from the positive electrode active material. In general, the positive electrode active material is a lithium compound including a transition metal such as nickel (Ni), cobalt (Co), and manganese (Mn), and when the positive electrode active material, which produces a large amount of gas produced during charging, is charged, it lose electrons and is reduced to increase oxidation power, and since the electrolyte solution is a solvent formed of carbonate as a main component, the charged positive electrode active material takes electrons from the surrounding electrolyte solution and oxidize carbonate to produce carbon dioxide and oxygen. FIG. 5 is a schematic diagram in which an electrolyte solution reacts with a charged positive electrode active material on the surface of the positive electrode active material to produce gas, and in more detail with reference to FIG. 5, when a Li+ ion is released from the positive electrode active material during the charge of the positive electrode active material, the transition metal is oxidized for adjusting electrical neutrality and donates an electron. Referring to FIG. 5, it is shown that a trivalent transition metal is charged and partly reduced to a tetravalent metal. The tetravalent transition metal receives an electron again to be reduced, and since it is considered that the tetravalent transition metal has a strong ability to take electrons from other materials, it is regarded as having high oxidation strength. When there is a material providing electrons, it takes electrons, and the affinity as such is stronger at a higher temperature. The electrolyte solution is formed of a lithium salt and a solvent, and the solvent is formed of a carbonate material. The solvent easily loses electrons and is oxidized on the surface of the charged positive electrode active material having high oxidation strength. Due to the oxidation of carbonate, carbon dioxide and oxygen occur. When a lithium secondary battery is allowed to stand at a high temperature or to stand in a charged state, a basic principle of producing gas in a positive electrode area is as described above, and in the present disclosure, the principle is used to measure the pressure of gas produced in the charged positive electrode.

Therefore, according to the present disclosure, the pressure of gas produced when the positive electrode active material is fixed and the kind of electrolyte solution or electrolyte solution additive is changed may be compared. As a factor affecting gas production, both the positive electrode active material and the electrolyte solution may be compared and evaluated.

An oven 1500 in which the gas analyzing apparatus 1000 is loaded may be further included. The oven 1500 may serve to control the temperature of the gas analyzing apparatus 1000, such as raising a temperature so that the positive electrode active material and the electrolyte solution react to produce gas and maintaining a specific temperature. In the present disclosure, the oven 1500 should be understood to refer to all means which may apply heat to the gas analyzing apparatus 1000.

FIG. 6 schematically illustrates a method of analyzing gas using the gas analyzing apparatus 1000 according to an exemplary embodiment in the present disclosure, and in the oven 1500, a plurality of gas analyzing apparatuses 1000 may be loaded, and the positive electrode active material and the electrolyte solution different from each other may be analyzed in the gas analyzing apparatus 1000, and the data such as pressure, temperature, and components measured herein may be sent to a data collection device 2000 connected to the outside of the oven 1500.

As such, the present disclosure provides an apparatus and method of quantitatively measure gas released by a reaction of the positive electrode active material and the electrolyte solution. According to the present disclosure, time and costs may be effectively reduced, and an amount of gas produced may be predicted.

A method of analyzing gas using the gas analyzing apparatus according to the present disclosure may include: retaining a positive electrode active material and an electrolyte solution; combining an upper plate and a lower plate of the gas analyzing apparatus and sealing a space between the upper plate and the lower plate; applying heat to the gas analyzing apparatus to produce gas from the positive electrode active material and the electrolyte solution; and measuring and collecting the gas pressure.

According to another aspect of the present disclosure, a positive electrode including the positive electrode active material is provided, and a secondary battery including: the positive electrode; a negative electrode; and a separator interposed between the positive electrode and the negative electrode is provided.

A secondary battery module may be formed by including the secondary battery according to the present disclosure as a unit cell, and also, one or more modules are packaged in a pack case to form a secondary battery pack. The secondary battery module and the secondary battery pack described above may be applied to various devices. The device may be applied to vehicles such as electric bicycles, electric automobiles, and hybrid cars, but the present disclosure is not limited thereto, and may be applied to various devices in which the secondary battery module and the secondary battery pack including the same may be used, and these also belong to the right scope of the present disclosure.

Hereinafter, the present disclosure will be described in detail through the specific examples. The following example is only illustrative for assisting in the understanding of the present disclosure, and the scope of the present disclosure is not limited thereto.

### EXAMPLES

### Examples 1 to 3

The gas analyzing apparatus of the present disclosure was used to measure a pressure of gas produced from three samples of NCM-based positive electrode active materials and electrolyte solutions different from each other, twice, respectively. More specifically, the sample of Example 1 was 63 mg of a first positive electrode active material powder and 30 ml of an electrolyte solution, the sample of Example 2 was 63 mg of a second positive electrode active material powder and 30 ml of an electrolyte solution, and the sample of Example 3 was 63 mg of a third positive electrode active material powder and 30 ml of an electrolyte solution. The electrolyte solutions were the same. Meanwhile, the first positive electrode active material powder and the second positive electrode active material used the same precursor and doping material and included 88 mol% of nickel, but the first positive electrode active material was coated, while the second positive electrode active material was not coated. The third positive electrode active material used a precursor and a doping material completely different from those of the first and second positive electrode active materials, and included 83 mol% of nickel.

### Comparative Examples 1 to 3

The NCM-based positive electrode active material and the electrolyte solution used in Examples 1 to 3 were used to manufacture secondary battery full-cells which were Comparative Examples 1 to 3, and the secondary battery full-cells were allowed to stand at a high temperature of 60° C. in a completely charged state, and a gas pressure and a gas amount produced at week 8 and week 20. The full-cells were manufactured by the following process.

### Positive Electrode

A positive electrode active material and a conductive material (Denka Black) binder (PVDF) were mixed at a mass ratio of 92:5:3 to prepare a positive electrode slurry, which was coated, dried, and rolled on an aluminum substrate to manufacture a positive electrode.

### Negative Electrode

A negative electrode active material (natural graphite (d002 3.358 Å)), a conductive material (KS6, plate type), a binder (SBR), and a thickening agent (CMC) were mixed at a mass ratio of 93:5:1:1 to prepare a negative electrode slurry, which was coated, dried, and rolled on a copper substrate to manufacture a negative electrode.

### Separator

As a separator, polyethylene having a thickness of 25 µm was used.

### Manufacture of Full-Cell

A positive electrode plate and a negative electrode plate were notched to an appropriate size, respectively and laminated, a separator was interposed between the positive electrode plate and the negative electrode plate to form a cell, and a positive electrode tab and a negative electrode tab were welded, respectively. A combined of welded positive electrode/separator/negative electrode was placed in a pouch, and three sides except an electrolyte solution injection side was the sealed. At this time, a portion where a tab exists was included on a sealing member. The electrolyte solution was injected to the remaining one portion, the remaining one side was sealed, and impregnation was performed for 12 hours or more. Thereafter, pre-charging with a current (2.5 A) corresponding to 0.25 C was performed for 36 min. After 1 hour, degassing was performed, aging was performed for 24 hours or more, and then formation charging and discharging were performed (charge condition: CC-CV 0.2 C 4.2 V 0.05 C CUT-OFF, discharge condition: CC 0.2 C 2.5 V CUT-OFF). Thereafter, standard charging and discharging were performed (charge condition: CC-CV 0.5 C 4.2 V 0.05 C CUT-OFF, discharge condition: CC 0.5 C 2.5 V CUT-OFF).

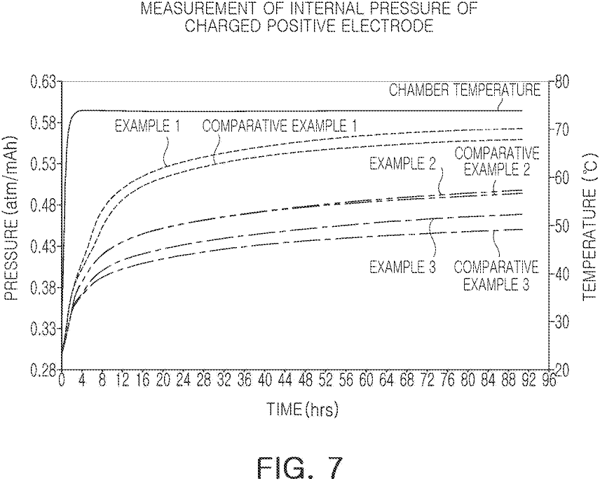

FIG. 7 shows a gas pressure measured in Examples 1 to 3, and the following Table 1 shows amounts of gas produced from the full-cells manufactured in Comparative Examples 1 to 3 at week 8 and week 20.

TABLE 1

Total amount of gas produced (ml) stored at 60° C.

| | Week 8 | Week 20 |
|---|---|---|
| Comparative Example 1 | 24.9 | 95.5 |
| Comparative Example 2 | 28.8 | 103.7 |
| Comparative Example 3 | 119.4 | 125.7 |

Referring to FIG. 7, it was confirmed that graphs of Examples 1 to 3 measured twice, respectively, showed similar numerical values and tendency. In addition, referring to FIG. 7 and Table 1, it was found that Examples 1 to 3 and Comparative Examples 1 to 3 had coincident amounts of gas produced and tendency. That is, according to the present disclosure, the results obtained by performing measurement from a full-cell for about 20 weeks or more were able to be obtained in 90 hours (about 3.7 days), and the results were also highly reliable.

In a conventional method which was used for analyzing gas produced inside a secondary battery, it is essential to actually manufacture a secondary battery cell, but the positive electrode active material according to the present disclosure allows prediction of an amount of gas produced and gas components in a secondary battery cell without actually manufacturing a secondary battery cell. In addition, a process from sample preparation to measurement completion, which is required for measuring an amount of gas produced may be performed within a short time.

In addition, when a positive electrode including a positive electrode active material having a predetermined pressure measured using the gas analyzing apparatus of the present disclosure is applied to a secondary battery, an amount of gas produced in an actual secondary battery may be expected, and thus, the performance such as life and high-temperature storage characteristics of a secondary battery may be guaranteed.

Hereinabove, the exemplary embodiments in the present disclosure were described in detail, however, the scope of a right of the present disclosure is not limited thereto, and it is apparent to a person skilled in the art that various modifications and changes are possible within the scope not departing from the technical idea of the present disclosure.

While exemplary embodiments have been shown and described above, it will be apparent to those skilled in the art that modifications and variations could be made without departing from the scope of the present disclosure as defined by the appended claims.

What is claimed is:

1. A positive electrode active material which has a pressure of gas produced by a reaction with an electrolyte solution of 0.4 to 0.6 atm/mAh.

2. The positive electrode active material of claim 1, wherein the reaction is performed at a temperature of 70 to 75° C.

3. The positive electrode active material of claim 1, wherein the positive electrode active material is collected from a half-cell charged with predetermined SOC.

4. The positive electrode active material of claim 1, wherein the positive electrode active material includes 80 mol% or more of nickel.

5. The positive electrode active material of claim 1, wherein a weight ratio between the positive electrode active material and the electrolyte solution is 1:1 to 3:1.

6. A positive electrode comprising the positive electrode active material of claim 1.

7. A secondary battery comprising: the positive electrode of claim 6; a negative electrode; and a separator interposed between the positive electrode and the negative electrode.

8. A gas analyzing apparatus comprising:
a lower plate including a retention portion in which an electrolyte solution and a positive electrode active material are retained;
an upper plate including a first flow path in which gas produced by a reaction of the electrolyte solution and the positive electrode active material moves and a pressure measuring sensor;
an internal pressure control port which controls opening and closing of a second flow path communicating with the first flow path of the upper plate and controls pressure inside the apparatus; and
a sealing member sealing a space between the upper plate and the lower plate.

9. The gas analyzing apparatus of claim 8, wherein the retention portion has a volume of 70 to 80 mm³.

10. The gas analyzing apparatus of claim 8, wherein an oven in which the gas analyzing apparatus is loaded is further included.

11. The gas analyzing apparatus of claim 8, further comprising a data collection port which collects data measured from the gas analyzing apparatus.

12. The gas analyzing apparatus of claim 8, further comprising a fastening member which fixes the upper plate and the lower plate.

13. The gas analyzing apparatus of claim 8, further comprising a gas chromatography analysis unit connected to the internal pressure control port.

14. The gas analyzing apparatus of claim 8, further comprising a temperature sensor which measure a reaction temperature of the electrolyte solution and the positive electrode active material.

15. The gas analyzing apparatus of claim 14, wherein the temperature sensor is formed in a lower surface of the upper plate adjacent to the retention portion.

16. The gas analyzing apparatus of claim 8, wherein the lower surface of the upper plate adjacent to the retention portion is formed in a dome structure.

17. The gas analyzing apparatus of claim 8, wherein the positive electrode active material is collected from a half-cell charged with predetermined SOC.
